(19) United States
(12) Patent Application Publication
(10) Pub. No.: US 2009/0087923 A1
(43) Pub. Date: Apr. 2, 2009

(54) METHOD OF HIGH SENSITIVE IMMUNOASSAY

(76) Inventors: Koji Muraya, Asaka-Shi (JP); Yoshio Saito, Asaka-Shi (JP)

Correspondence Address: BIRCH STEWART KOLASCH & BIRCH, PO BOX 747, FALLS CHURCH, VA, 22040-0747, US

(21) Appl. No.: 12/237,044

(22) Filed: Sep. 24, 2008

(30) Foreign Application Priority Data
Sep. 28, 2007 (JP) 2007-254178

(51) Int. Cl. G01N 33/566

(52) U.S. Cl. 436/501

(57) ABSTRACT

An object of the present invention is to provide a method of high sensitive immunoassay using immunoagglutination reaction by antigen-antibody reaction for quantification of thyroid stimulating hormone (TSH). The present invention provides a method of assaying a thyroid stimulating hormone (TSH) comprising assaying agglutination which is generated by contacting TSH with a carrier to which an anti-TSH antibody has been bound, wherein a plurality of types of anti-TSH antibodies that recognize different epitopes of TSH are independently supported on separate carriers, and each carrier on which a TSH antibody has been supported is brought into contact with a TSH-containing analyte with time intervals.

### TECHNICAL FIELD

The present invention relates to a method of immunoassay comprising performing an agglutination reaction using an immunological material such as an antigen or antibody, and a carrier such as latex, to thereby assay an antigen. More particularly, the present invention relates to a method of high sensitive immunoassay comprising allowing antithyroid stimulating hormone (TSH) antibodies to be individually supported on an individual carrier and allowing the antibodies to react with an analyte with time intervals.

### BACKGROUND ART

In the past, a disease has been identified through clinical diagnosis by sampling a body fluid component, such as blood (e.g., whole blood, blood plasma, or blood serum) or urine, using such body fluid component as an analyte, quantifying a concentration of a given component in the analyte, and employing the concentration of such given component as an indicator. When an assay target component in the analyte is an antigen or antibody in such clinical test settings, an immunodiagnostic reagent has been used as a means for quantifying the component with the utilization of properties of an antibody to strictly distinguish and bind to an antigen.

A representative example of an assay target component of such immunodiagnostic reagent is a hormone. A trace amount of hormone is present in the blood in vivo, and hormone is a substance that regulates a variety of physiological activities. Thyroid hormones, i.e., thyroxine (T4) and thyroid stimulating hormone, in particular, are often assayed in clinical settings, in order to determine and evaluate thyroid functions. Under normal conditions, the thyroid gland is stimulated by the thyroid stimulating hormone (TSH), and thyroxine (T4) is secreted. When thyroid functions are damaged, such as in the case of Hashimoto's disease, however, T4 would not be secreted, even though TSH is secreted. Accordingly, it is necessary to assay both concentrations of T4 and TSH in the blood, when a thyroid dysfunction is suspected.

In the blood, however, only a trace amount of hormones represented by TSH or T4 are present. This requires the use of a reagent and an apparatus that are capable of highly sensitive quantification. In order to realize assay of TSH and T4 in the POCT field, assay is required to be of high sensitivity, and at the same time, assay must be carried out in a cost-effective and simple manner.

When using the antigen-antibody reaction-based immunodiagnostic reagent, methods that do not involve the use of a labeling reaction, i.e., non-labeling methods, such as the precipitation reaction-based immunodiffusion, immunoturbidimetry, and immunonephelometry, the agglutination reaction-based hemagglutination or latex method and the like may be employed. To date, enzyme immunoassay (EIA), radioimmunoassay (RIA), fluorescence immunoassay (FIA), chemiluminescence immunoassay (CLIA), bioluminescence immunoassay (BLIA), and other techniques have been employed as methods that involve the use of a labeling reaction, in accordance with types or properties of materials to be labeled.

In the EIA method, for example, an enzyme reaction is allowed to proceed for a long period of time to accumulate (i.e., amplify) the reaction product, and a signal emitted from enzyme is then detected. Thus, a very sensitive immune-reactive reagent can be prepared. This applies to the other labeling techniques. Such technique, however, requires labeling of an antibody or antigen bound to a target antigen or antibody as an assay target component with an enzyme (e.g., peroxidase, β-galactosidase, or alkaline phosphatase). This complicates preparation of a reagent. Labeling techniques, including the ETA method, are generally classified into homogeneous techniques that do not involve B/F separation and heterogeneous techniques that involve B/F separation. The heterogeneous technique is particularly excellent in terms of sensitivity and safety because of B/F separation; however, the heterogeneous technique is inferior to the homogeneous technique in terms of promptness and convenience.

Further, labeling techniques, such as the EIA or RIA method, necessitate complicated assay operations and use of a special assay apparatus, equipment, facilities, and the like. Manufacturers of labeling-based immunodiagnostic reagents have overcome such drawbacks by supplying relatively large automated analyzers, which are developed exclusively for their own reagents. This consequently makes extensive-spreading of immunodiagnostic reagents and systems difficult in the POCT field.

Thus, labeling techniques are advantageous in terms of sensitivity and safety; however, such techniques are disadvantageous in terms of promptness and convenience. Further, labeling techniques necessitate complicated assay operations and use of a special assay apparatus, equipment, facilities, and the like. Accordingly, labeling techniques can be relatively easily employed at moderate- to large-scale hospitals equipped with a central laboratory where clinical technologists are always present. At small-scale medical facilities, such as small-scale hospitals, practitioners, clinics, or veterinary hospitals, or in the POCT field, however, employment of such techniques still remains difficult.

In the case of non-labeling techniques typified by the latex method, a process of labeling is unnecessary. Also, photometry at a given wavelength in the ultraviolet, visible, and near-infrared regions enables quantification of assay target components. Thus, provision of general and small spectroscopic apparatus is sufficient. Accordingly, it is easily deduced that employment of non-labeling techniques in the small-scale medical facilities or in the POCT field is easy.

While non-labeling techniques are advantageous in terms of promptness and convenience, the aforementioned amplification effects of the labeling techniques cannot be attained in principle. Thus, non-labeling techniques are disadvantageous in terms of sensitivity and safety. From a different point of view, this indicates that improvement in sensitivity of a non-labeling technique, such as the latex method, enables easy introduction of immunodiagnostic reagents and systems into small-scale medical facilities or the POCT field.

In particular, an agglutination reaction via the latex method is excellent in terms of promptness, convenience, and general versatility in the following respects. That is, a method for preparing a reagent is relatively easy. A user is only required to add an analyte to the latex reagent to initiate an assay, assay may be carried out at a given wavelength in the ultraviolet, visible, and near-infrared regions for a given period of time, and assay is generally completed within several minutes to several tens of minutes.

As a technique of supersensitization in conventional latex agglutination techniques, addition of a given sensitizer (agglutination promoter), such as polyvinyl pyrrolidone, polyethylene glycol, dextran, or dextran sulfate, disclosed in, for example, JP Patent Publication (kokai) No. H05-80051 A (1993), H05-297002 A (1993), or H06-58935 A (1994) is available. Although such sensitizer is effective for increasing assay sensitivity and a signal intensity, such sensitizer would also increase non-specific reactions disadvantageously. A method of defining diameters of latex particles to be used in a given range, a method of allowing the presence of latex particles having different diameters, and a method of adding a surfactant disclosed in JP Patent Publication (kokai) Nos. S60-53846 A (1985), H01-118770 A (1989), and H02-257063 A (1990) are disadvantageous in the following respect. As a latex particle diameter becomes larger, sensitivity is increased, but the range of quantification becomes narrower. In the presence of latex particles having different diameters, the effects of an individual particle diameter are limited. When a surfactant is to be added, the effects of sensitivity improvement are insufficient.

In the method of treating an antibody with a reducing agent to fragmentize the antibody and allowing the fragmented antibody to be supported on a carrier such as latex disclosed in JP Patent Publication (kokai) No. 2000-206115 A, pretreatment of an antibody is required. This sacrifices convenience of preparation of a reagent, which is an advantage of the latex agglutination method.

JP Patent Publication (kokai) No. H10-90268 A (1998) discloses an immunological particle agglutination reaction using both a polyclonal antibody and a monoclonal antibody as antibodies reacting with an assay target component. However, this technique is not intended for supersensitization, i.e., improvement in a response of an assay target component at a low concentration region. This technique is intended to inhibit the prozone phenomenon at a high concentration region and to enable assay in an extensive dynamic range even with the use of an apparatus without a mechanism of diluting an analyte.

### DISCLOSURE OF THE INVENTION

An object of the present invention is to provide a method for selectively and specifically enhancing the agglutination reaction of TSH as an assay target component using an anti-TSH antibody without deterioration of convenience of preparation of a reagent, which is an advantage of the latex agglutination method and which could not be attained via a conventional method of supersensitizing immunodiagnostic reagents.

The present inventors have conducted concentrated studies in order to attain the above object. As a result, they succeeded in selectively and specifically enhancing the agglutination reaction of TSH, which is an assay target component, while maintaining convenience of preparation of a reagent, by allowing a plurality of types of anti-TSH antibodies reacting with different epitopes to be individually supported on a carrier, and allowing the antibodies to react with the analyte with time intervals, at the time of TSH assay. This has led to the completion of the present invention.

Thus, the present invention provides a method of assaying a thyroid stimulating hormone (TSH) comprising assaying agglutination which is generated by contacting TSH with a carrier to which an anti-TSH antibody has been bound, wherein a plurality of types of anti-TSH antibodies that recognize different epitopes of TSH are independently supported on separate carriers, and each carrier on which a TSH antibody has been supported is brought into contact with a TSH-containing analyte with time intervals.

Preferably, the each carrier on which a TSH antibody has been supported is brought into contact with a TSH-containing analyte at a time intervals of 1 to 20 minutes.

Preferably, the anti-TSH antibody is a monoclonal antibody or a polyclonal antibody.

The present invention provides a method for assaying thyroid stimulating hormone (TSH) that selectively and specifically enhances the agglutination reaction of the assay target components while maintaining convenience of preparation of a reagent.

### BRIEF DESCRIPTION OF THE DRAWING

FIG. 1 shows a response of the TSH-antibody-bound latex that recognizes epitope A, B, or C to the standard TSH solution.

FIG. 2 shows the results of a comparison of changes in OD values at the initial reaction stage relative to the TSH concentration.

### BEST MODES FOR CARRYING OUT THE INVENTION

Hereafter, the present invention is described in detail.

The method of assaying the thyroid stimulating hormone (TSH) of the present invention comprises assaying agglutination which is generated by contacting TSH with a carrier to which an anti-TSH antibody has been bound. In this method, a plurality of types of anti-TSH antibodies that recognize different epitopes of TSH are independently allowed to be supported on separate carrier, and each carrier on which a TSH antibody has been supported is brought into contact with a TSH-containing analyte with time intervals. The term “assay of TSH” used herein should be understood in the broadest possible way, including detection of the presence or absence of TSH and assay of the TSH amount (i.e., quantification). Also, the term “assay of agglutination” used herein is not limited to assay of the degree of agglutination. It should be understood in a broad way, including, for example, observation of agglutination.

An anti-TSH antibody that can be used in the present invention may be a monoclonal antibody or a polyclonal antibody without particular limitation. So long as a plurality of types of antibodies are selected from antibodies that recognize different epitopes of TSH which is the assay target component, the antibody may be a monoclonal antibody or a polyclonal antibody.

Also, animal species of the anti-TSH antibody are not particularly limited. For example, a monoclonal antibody or a polyclonal antibody derived from a mouse, rat, rabbit, goat, sheep, or human can be used. Further, a modified antibody such as a chimeric antibody, a commercialized antibody, or an antibody prepared from the animal blood serum or culture supernatant in accordance with a conventional technique can be used.

The time at which a carrier comprising a plurality of types of anti-TSH antibodies supported thereon is allowed to react with an analyte, as well as the relevant time intervals, are not particularly limited, provided that such reactions are not simultaneously carried out. This enables more selective and specific enhancement of agglutination of TSH as an assay target component, compared with a case in which the reactions with the analyte are simultaneously carried out. A time interval is preferably 1 to 20 minutes, and more preferably 1 to 10 minutes.

In the present invention, known carriers can be used without particular limitation, provided that such carriers can support an antibody thereon, and the antibody-supporting carrier can cause the agglutination reaction resulting from an antigen-antibody reaction in the presence of an antigen. The amounts of antibodies and carriers used for preparing carriers on which antibodies are supported are not particularly limited, and any amounts can be employed.

Conventional polystyrene particles can be used as carrier. The material of such particle is not particularly limited, provided that such material is a known material for a carrier used in the field of immunoagglutination reaction. Examples of such materials include synthetic polymer particles, such as polystyrene, a styrene-methacrylic acid copolymer, a styrene-glycidyl (meth)acrylate copolymer, a styrene-styrene sulfonate copolymer, a methacrylic acid polymer, an acrylic acid polymer, an acrylonitrile-butadiene-styrene copolymer, a vinyl chloride-acrylic acid ester copolymer, and a polyvinyl acetate-acrylate copolymer. A latex suspension comprising such particles homogeneously suspended therein is preferable. Further examples include other organic polymer powders, inorganic powders, microorganisms, blood cells or pieces of cell membrane, and a plastic microtiter plate. Examples of inorganic powders include metal pieces of gold, titanium, and nickel, silica, and alumina.

The diameters of such carrier particles vary depending on the material of a carrier, the concentration range at which an assay target component is quantified, the assay equipment, the method, or the like. In general, a diameter is 0.01 to 1.0 μm, and more preferably 0.05 to 0.7 μm.

When preparing a reagent by allowing a carrier to support an antibody thereon, any known method for preparing a reagent for immunoagglutination as disclosed in, for example, JP Patent Publication (kokai) No. 2000-206115 A can be used. An antibody can be supported on a carrier via physical adsorption or chemical binding via covalent binding. After an antibody is supported on a carrier, a surface of a carrier that is not covered by an antibody can be coated with a known blocking agent, such as bovine serum albumin (BSA), Block Ace, skimmed milk, or casein. Such blocking agent may be subjected to pretreatment such as partial denaturation with heat, acid, alkali, or the like, according to need.

An example of a specific method for preparing a reagent by allowing a carrier to support an antibody thereon is described below. To a solution comprising latex particles dispersed at a solid concentration of 0.1% to 10% therein, an antibody solution adjusted at a concentration of 0.01 to 20 mg/ml is added, and the resultant is then mixed. The mixture is continuously agitated at 4° C. to 50° C. for 10 minutes to 48 hours. Thereafter, a solution is separated from carriers via centrifugation or other means to thoroughly remove antibodies that did not bind to carriers from the solution. Thereafter, carriers are repeatedly washed with buffer 0 to 10 times. Carriers are mixed with antibodies to bind the antibodies to the carriers, and part of the carrier surface to which no antibodies have been bound is then preferably protected with a component that is not associated with an antigen-antibody reaction, preferably a protein, and more preferably a blocking agent, such as bovine serum albumin (BSA), Block Ace, skimmed milk, or casein, for the purpose of preventing a non-specific reaction at the time of assay.

According to the method of the present invention, a plurality of types of anti-TSH antibodies that recognize different epitopes of TSH are independently allowed to be supported on a separate carrier, and each carrier on which a TSH antibody has been supported is brought into contact with a TSH-containing analyte with time intervals. Specifically, the first carrier comprises, supported thereon, the first anti-TSH antibody selected from among a plurality of types of anti-TSH antibodies reacting with different epitopes, and the second carrier comprises, supported thereon, the second anti-TSH antibody selected from among a plurality of types of anti-TSH antibodies reacting with different epitopes. When three or more types of antibodies are used, the third carrier can comprise, supported thereon, the third anti-TSH antibody. Such carriers comprising an anti-TSH antibody supported thereon can be stored in different containers.

Target analytes may be any fluid containing a biogenic component. Examples thereof that can be used include blood (whole blood, blood plasma, or blood serum), pleural fluid, ascites, lymph fluid, urine, feces, sweat, and spinal fluid. A biogenic component as an analyte target may be any substance, provided that an antigen-antibody reaction can be carried out. Examples thereof include immunoglobulin, another protein or peptide, a polysaccharide, a saccharide, lipid, hormone, hormone-like substance, and drug.

According to the assay method of the present invention, a dispersion of an antibody-supporting carrier is brought into contact with an analyte containing an assay target component to initiate the reaction, and generation of aggregates resulting from the antigen-antibody reaction is quantified by assay at a given wavelength in the ultraviolet, visible, or near-infrared region for a given period of time. In general, assay is completed within several minutes to several hours. The reaction may be initiated with the use of two solutions, i.e., a carrier dispersion and an analyte solution. Further, an additional solution containing a stabilizer or agglutination promoter may be prepared, and the reaction may proceed with the use of three solutions. Such stabilizer or agglutination promoter may be contained in a carrier dispersion or analyte solution, and such substance may be added according to need.

A stabilizer is not particularly limited, provided that it would stabilize the reagents and components used for the reaction or the progress of reaction. Examples thereof include a buffer, such as Good's buffer, a synthetic or natural polymer, such as polyethylene glycol or a polysaccharide, and a surfactant. This applies to a case in which such substance functions as a promoter of an agglutination reaction, as well as a stabilizer. The reagents and components used for the reaction refer to, for example, an antibody that has been bound to a carrier or an assay target component. As described in JP Patent Publication (kokai) No. H08-187095 A (1996), for example, addition of a saccharide for the purpose of stabilizing a protein such as an antibody or enzyme is known. Accordingly, copresence of a stabilizer, such as a saccharide or amino acid, for the purpose of stabilizing an antibody in the reagent, is easy to deduce. When an assay target component is an unstable substance such as a protein, a stabilizer may be allowed to be present for the purpose of maintaining the assay target stably during the assay.

Examples of an agglutination promoter include a synthetic or natural polymer, such as polyethylene glycol, polyglycosyl methacrylate, polyvinyl pyrrolidone, carboxy methyl cellulose, dextran, and pullulan. An example of a surfactant having similar effects is a polyethylene glycol fatty acid monoester derivative disclosed in JP Patent Publication (kokai) No. H05-297002 A (1993). A fatty acid constituting a hydrophobic portion may be saturated or unsaturated, and specific examples thereof include lauric acid, myristic acid, palmitic acid, stearic acid, arachic acid, behenic acid, oleic acid, linoleic acid, and linolenic acid.

A buffer that can be used may have a buffering capacity at a pH level at which a general antigen-antibody reaction is carried out. Such pH level is 5 to 11, and more preferably 6 to 10. Any buffer that can be used in general biochemical experiment, such as buffer capable of buffering at the required pH levels, can be used. Examples thereof include phosphate buffer, Tris buffer, MES buffer, HEPES buffer, glycine buffer, citrate buffer, and Good's buffer. Also, fluid associated with the agglutination reaction, such as a dispersion of antibody-supporting carriers or an analyte comprising an assay target component, can comprise a surfactant, a synthetic or natural polymer, an organic or inorganic reagent, or the like, according to need. For example, any known surfactant for an immunoagglutination reaction, such as a nonionic, anionic or cationic surfactant, can be used. The same applies to other reagents.

When an agglutination reaction resulting from an antigen-antibody reaction is to be performed, the degree of agglutination can be assayed visually, via filming with the use of a video or other means, or spectroscopically. Spectroscopic assay may be carried out in accordance with a known technique with the use of a solution comprising a dispersion of antibody-supporting carriers in contact with an analyte containing an assay target component. For example, diameters of carrier particles, concentration, or changes (increase or decrease) in the intensity of scattered light, absorbance, or transmitted beam with the elapse of a reaction time may be assayed.

Hereafter, an embodiment of a reagent for latex agglutination is described as an example of the present invention, although the technical scope of the present invention is not limited thereto.

### EXAMPLES

### Example 1

### Detection of Thyroid Stimulating Hormone (TSH)

### (1) Preparation of Antibody-Bound Latex

Seven clones of the commercially available anti-TSH monoclonal antibody (Medix) were purchased. These seven clones recognize any of three epitopes in the β-subunit of TSH. A general method for preparing monoclonal antibodies is described in, for example, Handbook of Experimental Immunology, (NAKASHIMA, Izumi (ed.), the University of Nagoya Press). Polystyrene latex particles (particle diameter: 0.5 μm, JSR) were diluted with glycine buffer (pH 9.6) to bring a solid concentration to 3%. In a similar manner, the seven clones of the anti-TSH antibody were adjusted at a concentration of approximately 1 mg/ml. The equivalent amounts of the seven clones were added to the latex dispersion, the resulting mixture was agitated, and the reaction was allowed to proceed at room temperature for 1 hour. Centrifugation was carried out at 11,000 rpm for 15 minutes, the supernatant was removed, and 0.2% BSA in glycine buffer (pH 9.6, containing 0.001% of Triton X-100) was added to the precipitate as a blocking agent to bring the solid latex concentration to 1.5%. After the resultant was agitated at room temperature for 1 hour, centrifugation was carried out at 11,000 rpm for 15 minutes, and the supernatant was removed. Glycine buffer (pH 9.6) was added to the precipitate for redispersion, centrifugation was carried out again at 11,000 rpm for 15 minutes, and the supernatant was removed. Glycine buffer (pH 9.6) was added to the precipitate for redispersion to a final solid latex concentration of 3.0% therein, and the resultant was designated as an anti-TSH antibody-bound latex reagent. As shown in Table 1, seven types of anti-TSH antibodies indicated by different clone numbers recognize any of three epitopes in the β-subunit of TSH. Specifically, clones Nos. 5401, 5404, and 5408 recognize the same epitope (A).

TABLE 1
Epitopes of anti-TSH antibodies

| | Clone No. | | | | | | |
|---|---|---|---|---|---|---|---|
| | 5401 | 5403 | 5404 | 5405 | 5407 | 5408 | 5409 |
| Epitope | A | B | A | C | C | A | B |

### (2) Detection of TSH

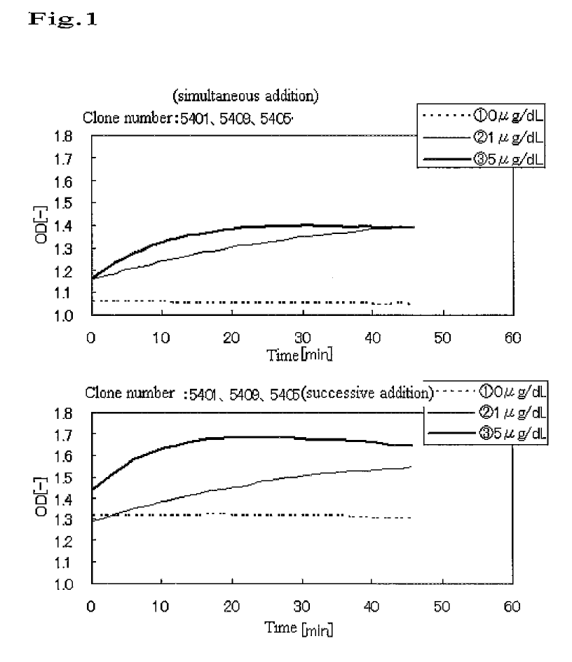

A standard TSH solution was diluted with buffer to bring the TSH concentration to 1.5 μg/dl, and the resultant was designated as a TSH solution (analyte). The anti-TSH antibody-bound latex shown in Table 1 was allowed to react with an analyte at 37° C., and changes in absorbance at given time points were assayed using a spectrophotometer (Hitachi, Ltd.). At the outset, 400 μl of the TSH solution diluted to a given concentration was added to the reaction cell, followed by preheating at 37° C. for 5 minutes. Thereafter, 600 μl of the anti-TSH antibody-bound latex reagent, which had been previously diluted to a final solid latex content of 0.015% in the reaction solution, was added to initiate the reaction, and the reaction was terminated 40 minutes after the initiation of the reaction. During the reaction, the absorbance at 800 nm was assayed every given period of time to determine the response to TSH at a given concentration (1.5 μg/dl) based on changes in absorbance. The results of assay (i.e., the time course of the agglutination reaction) are shown in FIG. 1. When initiating the reaction with the addition of the anti-TSH antibody-bound latex particles, a total of three types of latex particles were selected from among the latex particles to which antibodies of clones that recognize different epitope A, B, or C had been bound were selected. The case (i) where the reaction was initiated by simultaneously adding three types of latex particles and the case (ii) where the reaction was initiated by adding the first latex particle, the second latex particle five minutes thereafter, and the third latex particle five minutes thereafter were compared. As a result, the case (ii) where the latex particles were added with time intervals was found to show a greater initial rise and a more satisfactory response.

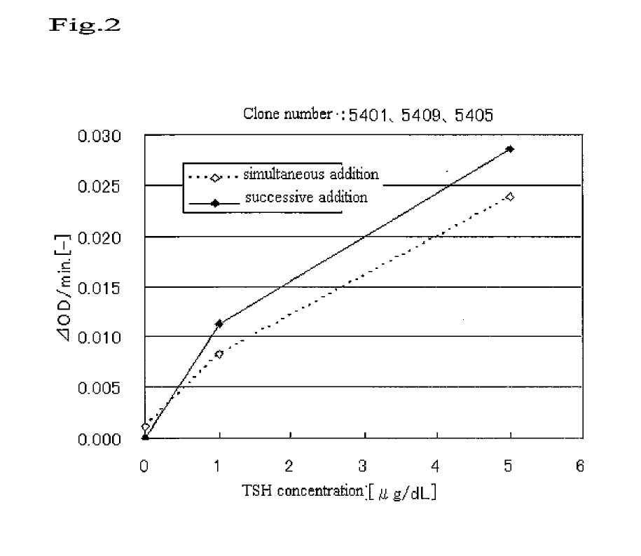

FIG. 2 shows the results of plotting the ΔOD/min. at the initial reaction stage relative to the TSH concentration (μg/dl) based on the time course shown in FIG. 1. A response at the TSH concentration of 1 μg/dL (3.6×10⁻¹⁰ M) was found to rise by approximately 40% when the latex particles were successively added, compared with the case when the latex particles were simultaneously added.

1. A method of assaying a thyroid stimulating hormone (TSH) comprising assaying agglutination which is generated by contacting TSH with a carrier to which an anti-TSH antibody has been bound, wherein a plurality of types of anti-TSH antibodies that recognize different epitopes of TSH are independently supported on separate carriers, and each carrier on which a TSH antibody has been supported is brought into contact with a TSH-containing analyte with time intervals.

2. The method of assaying TSH according to claim 1, wherein the each carrier on which a TSH antibody has been supported is brought into contact with a TSH-containing analyte at a time intervals of 1 to 20 minutes.

3. The method of assaying TSH according to claim 1, wherein the anti-TSH antibody is a monoclonal antibody or a polyclonal antibody.
